(19) United States
(12) Patent Application Publication
(10) Pub. No.: US 2021/0029956 A1
(43) Pub. Date: Feb. 4, 2021

(54) DIPLOID, FERTILE AND HIGHLY HOMOZYGOUS POTATO LINE SOLYNTUS

(71) Applicant: Agventure B.V., Wageningen (NL)

(72) Inventors: Willem Hendrik Lindhout, Wageningen (NL); Adriaan Willem van Heusden, Wageningen (NL); Ate van der Burgt, Wageningen (NL); Michiel Erik de Vries, Wageningen (NL); Su Ying, Wageningen (NL); Menno ter Maat, Wageningen (NL)

(21) Appl. No.: 16/941,998

(22) Filed: Jul. 29, 2020

Related U.S. Application Data
(60) Provisional application No. 62/879,769, filed on Jul. 29, 2019.

(51) Int. Cl. A01H 6/82; A01H 4/00; A01H 1/02

(52) U.S. Cl. CPC A01H 6/827; A01H 1/02; A01H 4/00

(57) ABSTRACT

The present invention relates to a diploid, fertile, self-compatible and highly homozygous tuber-bearing Solanum species potato line designated Solyntus. The potato line Solyntus produces 15 to 20 tubers per plant. The tubers have a yellow flesh. The phenotype of the plant and tuber is stable through both sexual and asexual reproduction. The potato line Solyntus is produced from seeds or by asexual reproduction of tubers or other plant parts.

### REFERENCE TO RELATED APPLICATION

This application claims the benefit of U.S. Provisional Application No. 62/879,769, filed Jul. 29, 2019, which is herein incorporated by reference in its entirety.

### FIELD OF INVENTION

The invention relates to sexually reproduced, fertile, self-compatible and highly homozygous diploid potato lines. More specifically, the invention relates to a diploid, fertile, self-compatible and highly homozygous Solanum species potato line designated Solyntus. The potato line Solyntus produces 15 to 20 tubers per plant. The tubers have a yellow flesh. The phenotype of the Solyntus plant and tuber is stable through either sexual or asexual reproduction.

### BACKGROUND OF INVENTION

The tuber-bearing potato Solanum species is the third largest human food crop in the world after wheat and rice. In 2017 worldwide potato production was more than 388 million metric tons. China and India are the largest potato producers.

Unlike other major field crops, potatoes are reproduced vegetatively (asexually) using potato tubers. The potato tubers used to produce new potato plants through vegetative reproduction are called “seed potato tubers.” Seed potato tubers are not produced sexually through production of sexually reproduced seed. Seed potato tubers are produced through asexual or vegetative propagation of tubers. Because potato is reproduced vegetatively (asexually), a significant portion of each year's crop is set aside for planting of seed potato tubers in the next planting season. About 5 to 15% of each year's crop is saved for replanting next year's crop instead of being used to feed people.

In the developing world, most farmers select and store their own seed potato tubers for plant of next year's crop. It is very common that these seed potato tubers are diseased and are infected with viruses, bacteria, and fungi. In developed countries, farmers are more likely to buy disease-free “certified seed potato tubers” from dedicated suppliers. The certified seed potato tubers are aseptically produced in vitro and therefore are less likely to transmit viruses, bacteria, fungi and nematodes to progeny. The production of certified seed potato tubers though vegetative propagation is expensive and the planting material is much too expensive for use in the developing world.

Seed potato tubers attract and transport pests and diseases. These pests and diseases include late blight, Andean potato weevils, nematodes, tuber moths and viruses. Viruses are transmitted in the field by aphids and then transmitted through vegetative propagation to seed tuber potatoes. Virus infection of potato can decrease yields by as much as 20 percent. Potato seeds that are produced by sexual reproduction do not transmit viruses and produce potato plants that are virus-free.

Another disadvantage of seed potato tubers is the high cost of transporting them. In developed countries, the disease-free seed potato tubers are frequently transported long diseases to the place where seed potato tubers are planted to produce the potato crop.

Yet another disadvantage of current seed potato tuber production methods, as well as current breeding methods, is that they rely on use of tetraploid potato plants. Conventionally made diploid potato tubers are generally too small for commercial applications. In addition, the tetraploid genome is extremely heterozygous, often containing multiple alleles per locus. In a typical progeny plant produced from the cross of two unrelated tetraploid parent lines, deleterious alleles may contribute to either reduced fitness in the case of homozygosity or increased vigor in the case of heterozygosity. Tetraploid breeding reduces the likelihood the breeder can combine the better alleles among the 30,000 genes in one progeny plant and therefore it is very difficult to create beneficial combinations of agronomically desirable traits.

Because such large populations of tetraploid progeny are needed to identify a potentially new potato selection with a new and attractive phenotype, the development of a new potato cultivar can often take up to eight years or more. In addition to the time required for breeding the new cultivar, five or more years will be needed to vegetatively propagate sufficient quantities of seed potato tubers for food production.

It is an objective of the present invention to provide a diploid, fertile, self-compatible potato plant that is highly homozygous, produces large quantities of tubers and can be propagated either sexually, through selfing and production of sexually-reproduced seeds, or asexually, through vegetative propagation of tubers. This potato plant line can be used to study the effects of homozygosity on plant growth and fertility in diploid, as well as developing inbred lines in a hybrid breeding program.

### SUMMARY OF INVENTION

In one aspect of the invention a diploid, fertile, highly homozygous and self-compatible tuber-bearing Solanum species line designated Solyntus is provided, representative seed of which has been deposited on 10 Jul. 2020 with the National Collection of Industrial and Marine Bacteria (NCIMB), Aberdeen, Scotland, UK, under NCIMB Accession No. 43636.

In another aspect of the invention, the diploid, fertile, highly homozygous and self-compatible tuber-bearing Solanum species plant line designated Solyntus is grown from seed having been deposited under NCIMB Accession No. 43636.

In another aspect of the invention the diploid, fertile, highly homozygous and self-compatible tuber-bearing Solanum species plant line designated Solyntus has a plant part that is suitable for sexual reproduction and that plant part is a seed, pollen or ovule.

In another aspect of the invention the diploid, fertile, highly homozygous and self-compatible tuber-bearing Solanum species plant line designated Solyntus has a plant part that is suitable for vegetative or asexual reproduction and that plant part is a tuber, a cell or a protoplast.

In yet another aspect of the invention a tissue culture of regenerable cells or protoplasts from the diploid, fertile, highly homozygous and self-compatible tuber-bearing Solanum species plant line designated Solyntus is provided.

In a further aspect of the invention is a method of producing a progeny plant of the diploid, fertile, highly homozygous and self-compatible tuber-bearing Solanum species potato plant line comprises (a) crossing the diploid, fertile self-compatible tuber-bearing Solanum species plant line designated Solyntus with itself, or with another diploid tuber-bearing Solanum species plant, (b) harvesting the resulting seed, and (c) growing said seed into said progeny plant.

Another aspect of the invention is a diploid progeny tuber-bearing Solanum species potato plant produced by (a) crossing the diploid, fertile, highly homozygous and self-compatible tuber-bearing Solanum species plant line designated Solyntus with itself, or with another diploid tuber-bearing Solanum species potato plant, (b) harvesting the resulting seed and (c) growing said seed into said progeny plant.

Yet another aspect of the invention is a method of producing a diploid tuber-bearing Solanum species plant line derived from diploid, fertile, highly homozygous and self-compatible tuber-bearing Solanum species line Solyntus, representative seed of which having been deposited under NCIMB Accession No. 43636, comprising the steps: (a) preparing a progeny plant derived from diploid, fertile, highly homozygous, self-compatible and tuber-bearing Solanum species line Solyntus by crossing Solyntus with itself or a second diploid and fertile tuber-bearing Solanum species plant; (b) crossing the progeny plant with itself or a second tuber-bearing Solanum species plant to produce a seed of a progeny plant of a subsequent generation; (c) growing a progeny plant of a subsequent generation from said seed and crossing the progeny plant of a subsequent generation with itself or a second diploid tuber-bearing Solanum species potato plant; and (d) repeating step (b) or (c) for at least 2 more generations to produce a diploid tuber-bearing Solanum species plant derived from the diploid tuber-bearing Solanum species line designated Solyntus.

A further aspect of the invention is a diploid tuber-bearing Solanum species plant derived from diploid, fertile, highly homozygous and self-compatible tuber-bearing Solanum species potato line Solyntus, representative seed of which having been deposited under NCIMB Accession No. 43636, comprising the steps: (a) preparing a progeny plant derived from diploid, fertile, highly homozygous and self-compatible tuber-bearing Solanum species line Solyntus by crossing a Solyntus plant with itself or a second diploid tuber-bearing Solanum species plant; (b) crossing the progeny plant with itself or a second tuber-bearing Solanum species plant to produce a seed of a progeny plant of a subsequent generation; (c) growing a progeny plant of a subsequent generation from said seed and crossing the progeny plant of a subsequent generation with itself or a second diploid tuber-bearing Solanum species plant; and (d) repeating step (b) or (c) for at least 2 more generations to produce a diploid tuber-bearing Solanum species plant derived from the diploid tuber-bearing Solanum species line designated Solyntus.

Another aspect of the invention is a method of producing a potato tuber from diploid, fertile, self-compatible tuber-bearing Solanum species line Solyntus, representative seed of which having been deposited under NCIMB Accession No. 43636 by (a) obtaining the plant Solyntus, (b) cultivating the plant to develop a tuber; and (c) collecting the tuber from the plant.

Yet another aspect of the invention is a tuber from the diploid, fertile, highly homozygous and self-compatible tuber-bearing Solanum species line Solyntus, representative seed of which having been deposited under NCIMB Accession No. 43636, produced by (a) obtaining the plant Solyntus, (b) cultivating the plant to develop a tuber; and (c) collecting the tuber from the plant.

A further aspect of the invention is a method of producing a seed of a Solyntus-derived diploid potato plant by (a) crossing a plant of diploid, fertile, highly homozygous and self-compatible tuber-bearing Solanum species line Solyntus, representative seed of which having been deposited under NCIMB Accession No. 43636, with itself or a second potato plant, and (b) obtaining seed of a Solyntus-derived potato plant.

An aspect of the invention is a method of producing a seed of a Solyntus-derived diploid potato plant by (a) crossing a plant of diploid, fertile, highly homozygous and self-compatible potato line Solyntus, representative seed of which having been deposited under NCIMB Accession No. 43636, with itself or a second potato plant, (b) obtaining seed of a Solyntus-derived potato plant, (c) crossing a plant grown from Solyntus-derived potato seed with itself or with a second diploid potato plant to yield Solyntus-derived potato seed, (d) growing additional Solyntus-derived potato seed of step (c) to yield additional Solyntus-derived potato plants, and (e) repeating the crossing and growing steps of (c) and (d) for an additional 1 to 10 generations to generate further Solyntus-derived potato plants.

Another aspect of the invention is a tuber-bearing Solanum species diploid, fertile, highly homozygous and self-compatible potato plant designated Solyntus, representative seeds of which having been deposited under NCIMB Accession No. 43636, further comprising a modification effected by transformation with a transgene, wherein said modified plant otherwise has all the phenotypic characteristics of the Solyntus diploid potato plant.

Another aspect of the invention is a tuber-bearing Solanum species diploid, fertile, highly homozygous and self-compatible potato plant designated Solyntus, representative seeds of which having been deposited under NCIMB Accession No. 43636, further comprising a modification effected by mutation or gene editing, wherein said modified plant otherwise has all the phenotypic characteristics of the Solyntus diploid potato plant.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

FIG. 1 shows Solynta plants grown in the greenhouse (A) and field (B). The plants in FIGS. 1A and 1B were grown from tubers.

FIG. 2A shows Solynta seedlings growing in the field. These seedlings were produced by selfing Solynta (F10), germinating the F10 seed in the greenhouse and transplanting the seedlings to the field.

FIG. 2B shows Solynta plants growing in the field that were produced by planting Solynta tubers.

FIG. 3A shows tubers produced by Solyntus grown in the greenhouse.

FIG. 3B shows tubers produced by F10 plants resulting from selfing of Solyntus and produced in the greenhouse.

### DEPOSIT

The deposit of Solyntus seed with the National Collection of Industrial and Marine Bacteria (NCIMB), Aberdeen, Scotland, UK, under NCIMB Accession No. 43636 was made and accepted pursuant to the terms of the Budapest Treaty. Upon issue of a patent, all restrictions upon the deposit will be removed, and the deposit is intended to meet the requirements of 37 CFR section 1.801-1.809. The deposit will be irrevocably and without restriction or condition release to the public upon the issuance of a patent and for the enforceable life of the patent. The deposit will be maintained in the depository for a period of 30 years and will be replaced if necessary during the period.

### DETAILED DESCRIPTION OF THE INVENTION

The following detailed description is given by way of example, but is not intended to limit the invention solely to the specific embodiments described.

### Definitions

The term “tuber-bearing potato” is used herein to refer to plant material that is essentially of the species Solanum tuberosum but may include introgression segments of other tuber-bearing Solanum species such as Solanum chacoense, Solanum phureja, Solanum andigena and Solanum demissum. Tuber-bearing Solanum species or Solanum is synonymous with the term potato.

The term “potato plant” is used herein to refer to a seedling or mature plant as grown from cell culture or seed. Persons of ordinary skill in the art will recognize that when the term “potato plant” is used in the context of the present invention, this also includes derivative lines or selections that retain the essential distinguishing characteristics of for instance potato plants that are the subject of the present invention such as a mutant or transgenic derivative having one or more additional genes incorporated into the genome. Backcrossing methods can also be used with the present invention to improve or introduce a characteristic into Solyntus.

The term “crossing” as used herein refers to the fertilization of female plants or gametes by male plants or gametes. The term “gamete” refers to the haploid reproductive cells, that is the egg or sperm, produced by plants by meiosis from a gametophyte and involved in sexual reproduction during which the egg and sperm fuse to form the diploid zygote. The term generally includes reference to pollen including the sperm cell and an ovule including the ovum. “Crossing” therefore generally refers to the fertilization of ovules of one individual with pollen from another individual, whereas “selfing” refers to the fertilization of ovules of an individual with pollen form the same individual. Crossing is widely used in plant breeding and results in a mix of genomic information between the two plants crossed because the progeny contain one chromosome from the female and the one chromosome from the male. This will result in a new combination of genetically inherited traits. Usually the progeny of a crossing are designated as “F1.” If the F1 is not uniform and segregates different phenotypes it is usually referred to as a F1 population. “Selfing” of a homozygous plant will usually result in a genetically identical plant since there is no genetic variation. “Selfing” of an F1 will result in an offspring that segregates for all traits that have heterozygotic loci in the F1. Such offspring are designated F2 or an F2 population.

When referring to “crossing” in the context of achieving the introgression of a genomic region or segment, the skilled person will understand that in order to achieve the introgression of only a part of a chromosome of one plant into the chromosome of another plant, it is required that random portions of the genomes of both parental lines will be recombined during the cross due to the occurrence of crossing-over events in the production of the gametes in the parent lines. Therefore, the genomes of bother parents must be combined in a single cell by a cross, where after the production of gametes from said cell, and their fusion during fertilization, will result in an introgression event.

As used herein, the terms “introgressing”, “introgress”, and “introgressed” refer to both a natural and artificial process whereby individual genes or entire chromosomes are moved from one individual plant, species, variety, cultivar, selection or line, into the genome of another individual plant, species, variety, cultivar, selection or line. In plant breeding, the process usually involves selfing or backcrossing to the recurrent parent to provide for an increasingly homozygous plant having essentially the characteristics of the recurrent parent in addition to the introgressed gene or trait.

The term “backcross” refers to the result of a “backcrossing” process wherein the plant resulting from a cross between tow parental lines is repeatedly crossed with one of its parental lines, wherein the parental line used in the backcross is referred to as the recurrent parent. Repeated backcrossing results in replacement of genome fragments of the donor parent with those of the recurrent patent. The offspring of a backcross is designated “BCx” or “BCx population”, where “x” stand for the number of backcrosses.

The term “selfing” refers to the process of self-fertilization wherein an individual is pollinated or fertilized with its own pollen. Repeated selfing eventually results in homozygous offspring.

As used herein, the term “line” refers to a population of plants derived from a single cross, backcross or selfing. The individual offspring plants are not necessarily genetically and phenotypically identical to one another but any variability can be properly described and the degree of homozygosity determined.

As used herein, the term “allele” means any of one or more alternative forms of a gene, all of which relate to at least one trait or characteristic. In a diploid cell or organism, the two copies of a gene occupy the corresponding loci on a pair of homologous chromosomes. Each copy may be a distinct allele.

A “gene” is defined herein as a hereditary unit that occupies a specific location on a chromosome and that contains the genetic instruction for a contribution to the potential phenotypic characteristics or trait in a plant.

A “locus” is defined herein as the position that a given gene occupies on a chromosome of a given plant species.

As used herein, the term “homozygous” means a genetic condition existing when identical alleles reside at corresponding loci on homologous chromosomes.

The term “essentially homozygous” refers to a level of homozygosity of at least 25%, preferably at least 50%, more preferably at least 75%, still more preferably at least 76, 77, 78, 79, 80, 81, 82, 83, 84, 85, 86, 87, 88, 89, 90, 91, 92, 93, 94, 95, 96, 97, 98, 99 or 100%, when testing 100, preferably 1,000, more preferably at least 10,000 loci. The skilled person will appreciate that the level of homozygosity of a plant is by definition the level of homozygosity as displayed across the whole genome of the plant, and that such testing of 100, preferably 1000, more preferably at least 10,000 loci reflects the level of homozygosity across the plant's genome, such as for instance obtained by random selection of loci. Homozygosity levels are average values for the population, and refer preferably to those loci wherein the parents differ. The term “highly homozygous” refers to a level of homozygosity of at least 75% when testing at least 1,000 loci.

As used herein, the term “heterozygous” means a genetic condition existing when different alleles reside at corresponding loci on homologous chromosomes

As used herein, the term “progeny” means genetic descendant or offspring.

The term “population” refers to a genetically heterogeneous collection of plants sharing a common features that are distinct from other populations.

As used herein, the term “hybrid” means any offspring of a cross between two genetically unlike individuals. More preferably the term “hybrid” refers to the cross between tow elite or inbred breeding lines.

The term “breeding line”, as used herein, refers to a line of a cultivated potato having commercially valuable or agronomically desirable characteristics, as opposed to a wild varieties or landraces.

As used herein, the term “elite line” refers to any line that has resulted from breeding and selection for superior agronomic performance. An elite line preferably is a line that has multiple desirable agronomic traits.

The terms “cultivar” or “variety” are used interchangeably herein and denote a plant which has deliberately been developed by breeding, crossing and selection, for the purpose of being commercialized, that is, used by farmers and growers to produce agricultural products. The term “breeding germplasm” denotes a plant having added value for commercial application and a biological status other than a “wild” status. Wild status indicates the plant is non-cultivated.

As used herein, the term “molecular genetic marker” or “marker” refers to an indicator that is used in methods for visualizing difference in the characteristics of nucleic acid sequences. Examples of such indicators are restriction fragment length polymorphism (RFLP) markers, amplified fragment length polymorphism (AFLP) markers, single nucleotide polymorphisms (SNPs), insertion/deletion (INDEL) mutations, microsatellite markers (SSRs), sequence-characterized amplified regions (SCARs), cleaved amplified polymorphic sequences (CAPs) markers or isozyme markers or combinations of the markers described herein which defines a specific genetic and chromosomal location. It is preferred to use Kasper as the SNP marker and SeqSNP as the sequence-derived marker.

Unless expressly stated otherwise, the term “seed”, as used throughout this specification refers to the body form which a new plant develops containing the small embryonic plant, usually together with some stored food, and enclosed in a seed coat. This seed, referred to as botanical or “true” seed is the product of the ripened ovule of gymnosperm and angiosperm plants which occurs after fertilization and some growth within the mother. In contrast, the term “seed tuber potatoes” refers to all, or a portion, of potato tuber that is used as the starting material for vegetative or asexual reproduction of a potato plant.

The term “diploid” as used herein refers to a plant wherein each vegetative cell contains two sets of chromosomes. (2x=2n wherein n is the numbers of chromosomes) One set of chromosomes is donated from each parent.

The term “tetraploid” as used herein refers to a plant wherein each vegetative cell contains four sets of chromosomes (2x=4n)

As used herein, the term “tuber flesh color” refers to the color of the interior of the tuber flesh after harvest as the result of the absence or presence of carotenoid compounds causing white or yellow flesh color, respectively. Tuber flesh color also encompasses a description of anthocyanin compounds that may cause red, blue, purple shades of flesh color in different patterns.

The term “fertile plant” is defined herein as a plant capable of producing fertile seed carrying berries. Preferably said berries each carry at least 5, more preferably at least 15-20, even more preferably at least 50 fertile seed, still more preferably between 50 and 500 seeds.

The term “self-compatible” refers to the capacity of a potato plant to develop viable seeds in berries that are the result of self-pollination or self-fertilization, that produce fertile progeny plants.

As used herein, the term “plant or part thereof” means any complete or partial plant, single cells and cell tissues such as plant cells that are intact in plants, cell clumps and tissue cultures from which potato plants can be regenerated, Examples of plant parts include, but are not limited to, single cells and tissues from pollen, ovules, leaves, embryos, roots, root tips, anthers, flowers, fruits, stems, shoots, scions, rootstocks, seed, protoplasts, calli, and the like.

### DESCRIPTION

The invention provides methods and compositions relating to plants, seeds, tubers, and derivatives of a new diploid, fertile self-compatible and highly homozygous potato line herein referred to as Solyntus that is uniform and distinct from other such potato plants, and may be stably produced through either vegetative (asexual) reproductive of seed tuber potatoes or through sexual reproduction by selfing of Solyntus plants and sexual reproduction of seed. Representative seed of Solyntus are deposited under NCIMB Accession No. 43636.

The development of potato hybrids is a major breakthrough in commercial breeding and production of potato. For the first time, diploid, fertile and highly homozygous potato hybrid plants exhibiting heterosis can be produced. Because viruses are not transmitted through sexually reproduced seed, it is more efficient to produce disease-free potato through sexual reproduction. Furthermore, diploid and fertile potato lines speed the rate at which potato breeding can progress. Finally, sexually reproduced seed is much less costly to transport than asexually or vegetatively propagated seed tuber potatoes.

Although commercial tetraploid potato varieties are fertile and self-compatible, their selfed progeny exhibit very significant inbreeding depression, that is, the selfed progeny do not grow vigorously, produce deformed tubers and produce few viable seeds. Likewise, prior art diploid and essentially homozygous potato lines also do not grow vigorously and produce few viable seeds.

Complexities in the inheritance of any trait influence the choice of breeding method. Backcross breeding is used to transfer one or a few favorable genes coding for a highly heritable trait into a desirable line. Various recurrent selection techniques are used to improve are used to improve quantitatively inherited traits controlled by numerous genes.

The development of potato hybrids involves development of highly homozygous, fertile and diploid parental lines, the crossing of these lines and the evaluation of the crosses. Pedigree breeding and recurrent selection breeding methods are used to develop cultivars from the breeding populations. Breeding programs combine desirable traits from two or more varieties, or more broad-based sources, into breeding pools from which lines are developed by selfing and selection of desirable phenotypes in highly homozygous backgrounds. The diploid, fertile and highly homozygous potato lines are crossed, and the hybrids from these crosses are evaluated to determine which have the desirable characteristics of interest.

With the availability of diploid, fertile and self-compatible potato plants, pedigree breeding may now be used for the improvement and development of inbred lines. Two parents which possess favorable and complementary traits are crossed to produce an F1 population of plants. A F2 population of plants is produced by selfing one or more F1 plants or by intercrossing selected F1 plants. Selection of the best progeny plants may begin in the F2 or F3 populations.

With the availability of diploid, fertile and self-compatible potato plants, mass and recurrent selections may now be used to improve the traits in a population of genetically diverse potato plants. A genetically diverse population of heterozygous individuals is either identified or created by intercrossing several different parents. The best plants are selected based on the expression of traits of interest. The selected plants are intercrossed to produce a new population of potato plants in which further cycles of selection are continued.

With the availability of diploid, fertile and self-compatible potato plants, backcross breeding may now be used to transfer genes for a simply inherited into a desirable homozygous potato cultivar or line that serves are the recurrent parent. The source of the trait to be transferred is called the donor plant. After the initial cross, individual progeny possessing the phenotype of the donor parent for the preferred trait are selected and repeatedly backcrossed to the recurrent parent. At the end of backcrossing, the new cultivar or line will have the phenotype of the recurrent parent and the desirable trait of the donor parent.

In addition to determining the phenotype of diploid progeny potato plants, the genotype of the plants and the degree of homozygosity of a plant, can be determined. There are many techniques available for analysis of genotype and homozygosity including Randomly Amplified DNAs (RAPDs), DNA Amplification Fingerprinting (DAF), Simple Sequence Repeats (SSRs), Restriction Fragment Length Polymorphisms (RFLPs) and the like. Single Nucleotide Polymorphisms (SNPs) are randomly distributed across genomes and are powerful tool for plant genotyping and determination of homozygosity.

A preferred method of genetic phenotyping is competitive allele specific PCR (KASP) analysis was performed by VHLgenetics (Wageningen, The Netherlands) using KASP assays designed to be specific for SNPs that segregate in the Solyntus genome. KASP assays were conducted according to the protocol supplied by the manufacturer (LGC Genomics GmbH, Berlin, Germany). The results from the KASP assays were visualized using SNPviewer (available at lgcgroup.com/products/genotyping-software/snpviewer) to confirm correct segregation and genotype calling.

With the availability of diploid, fertile and self-compatible potato plants, marker-assisted breeding can be employed to expedite the breeding process. For example, molecular markers closely linked to alleles of interest may be used to select plants that contain the alleles of interest during a backcrossing breeding program. The molecular markers may also be used to select toward the genome of the recurrent parent and away from the molecular markers from the donor parent.

With the availability of diploid, fertile and self-compatible potato plants, mutation breeding may now be employed to introduce new traits into potato cultivars, varieties and lines. Several chemical or physical treatments are known to the person skilled in the art which can be used to induce genetic mutations in plant species like lettuce. Mutagenesis may comprise the random introduction of at least one modification by means of one or more chemical compounds, such as ethyl methanesulphonate (EMS), nitrosomethylurea, hydroxylamine, proflavine, N-methyl-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast-neutron exposure, X-rays, gamma irradiation, and/or by insertion of genetic elements, such as transposons, T-DNA, retroviral elements.

Mutagenesis also may comprise the more specific, targeted introduction of at least one modification by means of homologous recombination, oligonucleotide-based mutation induction, zinc-finger nucleases (ZFNs), transcription activator-like effector nucleases (TALENs) or Clustered Regularly Interspaced Short Palindromic Repeat (CRISPR) systems.

With the availability of diploid, fertile and self-compatible potato plants, it is now possible to introduce a transgene into a potato cell or plant by means of transformation methods well known in to person of skill. Furthermore, it is possible to introgress the transgene into diverse potato plants using the methods described herein.

The invention provides a food or feed product comprising or consisting of a tuber or part thereof. The food or feed product may be fresh or processed, including but not limited to dried, frozen, powdered, chopped, cooked, roasted, fried, broiled, steamed, canned, or blanched.

### EXAMPLES

### Development of Solyntus

The diploid, fertile, highly homozygous, self-compatible tuber-bearing Solanum species potato line is derived from breeder's reference AGVD3, representative seeds of which have been deposited on Oct. 23, 2009, with the NCIMB, Aberdeen, Scotland, under Accession No. NCIMB 41665. AGVD3 is disclosed in U.S. Patent Publication No. 20120284852, which is incorporated herein by reference, and is the source of the self-compatibility gene Sli. AGVD3 represents a BC1 population obtained by crossing a plant of parent IVP07-10014 with a plant of parent D1, backcrossing the resulting F1 offspring plants with the IVP07010014 parent. It is estimate that AGVD3 is about 75% homozygous for the loci for which parents differ.

Genetically distinct F2 inbred plants carrying the Sli gene were crossed and the F1 SOLO12-1510 was selected. SOLO12-1510 was selfed over 9 generations and plant selections made. From the F9 population a plant designated 17BL5241-2004 was selected and designated Solyntus. Seed of diploid, fertile, highly homozygous and self-compatible tuber-bearing Solanum species potato line Solyntus were deposited as NCIMB Accession No. 43636.

The deposit of Solyntus seed with the National Collection of Industrial and Marine Bacteria (NCIMB), Aberdeen, Scotland, UK, under NCIMB accession No. 43636 was made pursuant to the terms of the Budapest Treaty. Upon issue of a patent, all restrictions upon the deposit will be removed, and the deposit is intended to meet the requirements of 37 CFR section 1.801-1.809. The deposit will be irrevocably and without restriction or condition release to the public upon the issuance of a patent and for the enforceable life of the patent. The deposit will be maintained in the depository for a period of 30 years and will be replaced if necessary.

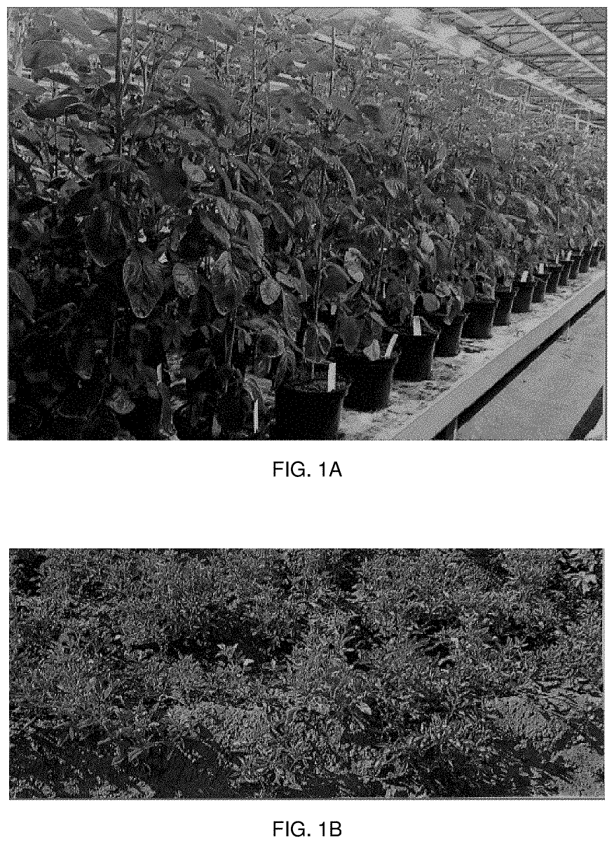

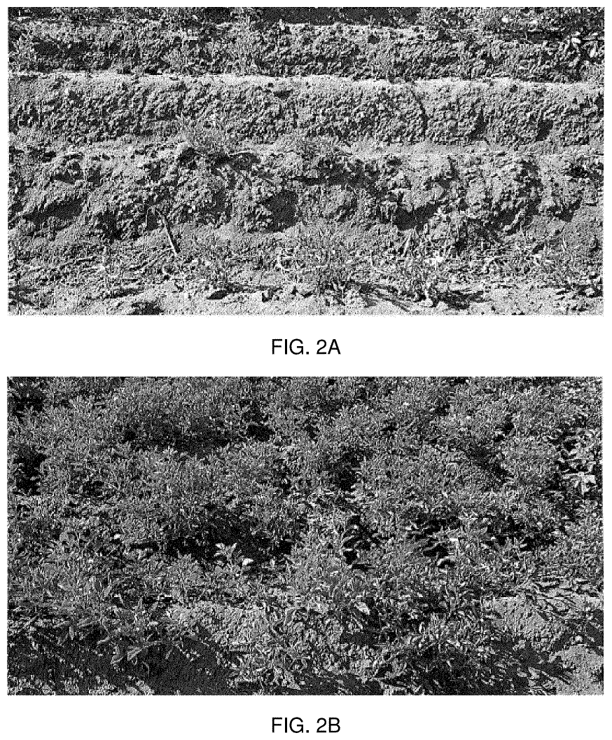

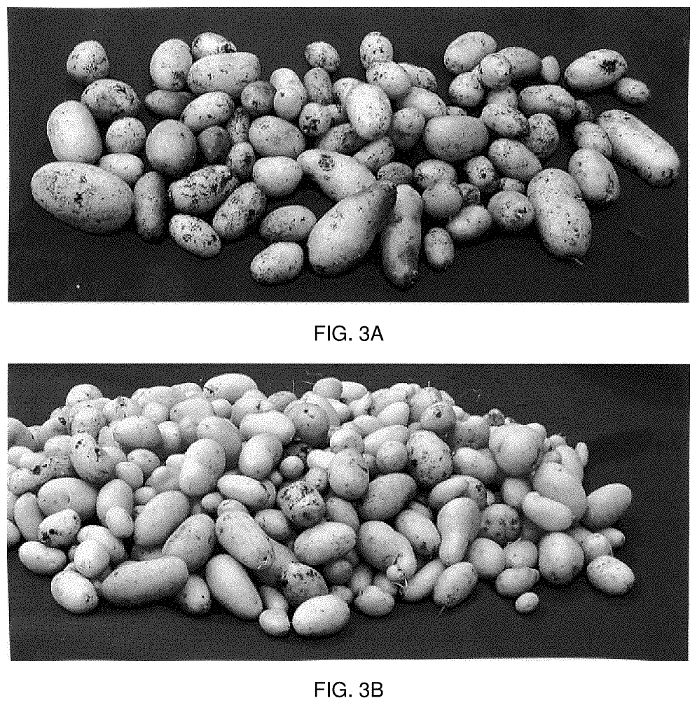

FIG. 1 shows Solynta plants grown in the greenhouse (A) and field (B). The plants in FIGS. 1A and 1B were grown from tubers. FIG. 2A shows seedlings growing in the field. These seedlings were produced by selfing Solynta (F10), germinating the F10 seed in the greenhouse and transplanting the seedlings to the field. FIG. 2B shows Solynta plants growing in the field that were produced by planting Solynta tubers. FIG. 3A shows tubers produced by Solyntus grown in the greenhouse. FIG. 3B shows tubers produced by F10 plants resulting from selfing of Solyntus.

Solyntus was determined to be 78.6% homozygous. The nucleotide sequence of the Solynta genome was determined Oxford Nanopore Technology and IIlumina sequencing. Long reads were used to place contigs in the right locations and short reads were used to fill in the gaps.

Important genetic physiological and morphological characteristics of Solyntus are summarized in Table 1.

TABLE 1
Genetic, Physiological and Morphological
Characteristics of Solyntus

Genetic Characteristics
78.6% homozygosity
Physiological and Morphologic Characteristics
Tuber
Average tuber yield per plant: 15 to 20
Tuber flesh color: light yellow
Fertility
Fertile producing flowers with viable seed.

1. A diploid, fertile, highly homozygous and self-compatible tuber-bearing Solanum species potato plant line designated Solyntus, representative seed of which has been deposited under NCIMB Accession No. 43636.

2. The plant of claim 1, wherein said plant was grown from seed having been deposited under NCIMB Accession No. 43636.

3. A part of the plant of claim 1, wherein said part of the plant is suitable for sexual reproduction.

4. The part of the plant of claim 3, wherein said part comprises a seed, pollen, or an ovule.

5. A part of the plant of claim 1, wherein said part of the plant is suitable for vegetative propagation.

6. The part of the plant of claim 5, wherein said part comprises a tuber, a cell or a protoplast.

7. A tissue culture of regenerable cells or protoplasts from the plant of claim 1.

8. A method of producing a progeny plant of the tuber-bearing Solanum species potato plant line of claim 1, comprising: (a) crossing the plant of claim 1 with itself, or with another diploid tuber-bearing Solanum species potato plant, (b) harvesting the resulting seed and (c) growing said seed into said progeny plant.

9. A diploid progeny potato plant produced by the method of claim 8.

10. A method of producing a diploid tuber-bearing Solanum species potato plant derived from diploid, fertile, highly homozygous and self-compatible tuber-bearing Solanum species line Solyntus, representative seed of which having been deposited under NCIMB Accession No. 43636, comprising the steps: (a) preparing a progeny plant derived from diploid, fertile, highly homozygous and self-compatible tuber-bearing Solanum species line Solyntus by crossing the plant of claim 1 with itself or a second diploid tuber-bearing Solanum species potato plant; (b) crossing the progeny plant with itself or a second tuber-bearing Solanum species potato plant to produce a seed of a progeny plant of a subsequent generation; (c) growing a progeny plant of a subsequent generation from said seed and crossing the progeny plant of a subsequent generation with itself or a second diploid tuber-bearing Solanum species potato plant; and (d) repeating step (b) or (c) for at least 2 more generations to produce an inbred diploid tuber-bearing Solanum species potato plant derived from the diploid, fertile, highly homozygous and self-compatible tuber-bearing Solanum species line designated Solyntus.

11. A diploid tuber-bearing Solanum species potato plant produced by the method of claim 10.

12. A method of producing a Solanum species potato tuber comprising: (a) obtaining the plant according to claim 1, wherein the plant has been cultivated to develop a tuber; and (b) collecting the tuber from the plant.

13. A tuber produced by the method of claim 12.

14. A method of producing a seed of a Solyntus-derived diploid potato plant comprising: (a) crossing the plant of claim 1, with itself or a second potato plant, and (b) obtaining seed of a Solyntus-derived potato plant.

15. The method of claim 14, further comprising (c) crossing a plant grown from Solyntus-derived potato seed with itself or with a second diploid potato plant to yield Solyntus-derived potato seed, (d) growing additional Solyntus-derived potato seed of step (c) to yield additional Solyntus-derived potato plants, and (e) repeating the crossing and growing steps of (c) and (d) for an additional 1 to 10 generations to generate further Solyntus-derived potato plants.

16. The tuber-bearing Solanum species of claim 1, further comprising a modification effected by transformation with a transgene, wherein said modified plant otherwise has all the phenotypic characteristics of the Solyntus diploid potato plant.

17. The tuber-bearing Solanum species of claim 1, further comprising a modification effected by mutagenesis, wherein said modified plant otherwise has all the phenotypic characteristics of the Solyntus diploid potato plant.

18. The tuber-bearing Solanum species diploid potato plant of claim 17, wherein the step of mutagenesis involves chemical mutagenesis or site-directed mutagenesis.
